(19) United States
(12) Patent Application Publication
(10) Pub. No.: US 2006/0160218 A1
(43) Pub. Date: Jul. 20, 2006

(54) Transdifferentiation of cells and tissues

(76) Inventors: Jonathan Slack, Bath (GB); Marko Horb, Montreal (CA); David Tosh, Bath (GB)

Correspondence Address: Leon R Yankwich;Yankwich & Associates, 201 Broadway, Cambridge, MA, 02139, US

(21) Appl. No.: 10/508,353

(22) PCT Filed: Mar. 17, 2003
(86) PCT No.: PCT/GB03/01180

§ 371 Date: Dec. 21, 2005

(30) Foreign Application Priority Data
Mar. 18, 2002 (GB) 0206357.6

(51) Int. Cl. C12N 15/87; C12N 15/86; C12N 5/08

(52) U.S. Cl. 435/455; 435/456; 435/366

(57) ABSTRACT

Method for carrying out transdifferentiation of non-pancreatic cells to pancreatic cells involving the provision of a pancreas specific transcription factor and an activating means such as VP16 of Herpes simplex virus to non-pancreatic cells. The methods and materials provided by the invention may be used to treat pancreatic disorders, in particular disorders caused by a loss of properly functioning pancreas such as pancreatic cancer and diabetes.

The present invention relates to the conversion of cells of one tissue type to pancreatic tissue, and materials for use therein, as well of the use of the method to produce pancreatic tissue that may be used as a potential treatment for pancreatic diseases such as diabetes and pancreatic cancer.

The pancreas arises from the endoderm as a dorsal and a ventral bud, which fuse together to form the single organ containing two distinct populations of cells, the exocrine cells that secrete enzymes into the digestive tract and the endocrine cells that secrete hormones into the bloodstream.

The exocrine pancreas is a lobulated, branched, acinar gland with secretory cells grouped into pyramidal acini with basal nuclei, regular arrays of rough endoplasmic reticulum, a prominent Golgi complex and numerous secretory (zymogen) granules containing secretory enzymes. At the junction of acini and ducts are low cuboid centroacinar cells, rich in mitochondria, that are thought to secrete non-enzymic components of the pancreatic juice, including bicarbonate. The ducts proper are lined with columnar epithelial cells, and in the larger ducts are found small numbers of goblet and brush cells similar to those of the intestine. The acini and smaller ducts are invested with a delicate, loose connective tissue, which becomes more extensive around the larger ducts.

The endocrine cells of the pancreas are mainly grouped into the islets of Langerhans, which are compact spheroidal clusters embedded in the exocrine tissue. There are four principal types of endocrine cell: the beta (or B) cells secrete insulin and also an insulin antagonist called amylin and make up the majority of the cells in the islets, the alpha (or A or A2) cells secrete glucagon, the delta (or D or A1) cells secrete somatostatin and the PP (or F) cells secrete pancreatic polypeptide.

Insulin is a dimeric SS (disulphide) linked protein that is synthesised as a single chain precursor which first loses its signal peptide, then loses a segment known as the C-peptide, before becoming the mature hormone molecule. The mature insulin is stored in secretory granules and its release is controlled by the level of glucose in the perfusing blood. The effects of insulin on the target tissues are both metabolic, particularly in promoting glucose uptake, and mitogenic.

In addition to the glandular components, the pancreas has a rich blood supply, the arterial blood passing in each lobule first to the islets and then to the adjacent acini. There is also an extensive lymphatic drainage and a rich sympathetic and parasympathetic nerve supply. Smooth muscle is found around the larger ducts and in the sphincter muscles of the two ampullae. The fibroblastic, lymphatic and smooth muscle components of the pancreas are presumed to arise from the abundant mesenchyme enveloping the embryonic buds.

The pancreas is a particularly important organ from the point of view of human medicine because it suffers from two important diseases: diabetes mellitus and pancreatic cancer. Diabetes affects at least 150 million people worldwide and despite the availability of insulin remains a major problem. Pancreatic cancer causes about 6500 deaths per annum in the UK and is virtually incurable. The pancreas is a particularly important tissue as it produces pancreatic hormones to control circulating fluid levels of glucose. When, after a meal, blood glucose rises above its normal level of 80 to 90 mg per 100 ml, insulin is released into the blood from secretory vesicles in the B cells in the islets of Langerhans of the pancreas. The islet cells themselves respond to the rise in level of glucose or amino acid levels by releasing insulin into the blood, which transports it throughout the body. By binding to cell surface receptors, insulin causes removal of glucose from the blood and its storage as glycogen. If glucose falls below about 80 mg per 100 ml, then the A cells of the islets begin secreting glucagon. The glucagon binds to a glucagon receptor on liver cells, activating adenylate cyclase and the cAMP cascade. The result is the degradation of glycogen and the release of glucose into the circulation.

There are two main types of diabetes mellitus. In type 1 or insulin-dependent diabetes, found most often in children and young people, the beta cells are destroyed by an autoimmune reaction and severe permanent insulin deficiency results. Type 2 or non-insulin dependent diabetes, more often found in older people, is a more complex and heterogeneous range of conditions, usually involving a degree of insulin non-responsiveness in the target tissues. In some cases there is a contribution from pancreatic pathology.

As pancreatic diseases are common, and yet the pancreas is essential for glucose metabolism, it is important to provide a system to replace pancreatic tissue which is missing or has become damaged, for example as a result of disease, surgery or otherwise.

To design a replacement or adjunct to the pancreas it is important to have a basic understanding of the cellular and molecular mechanisms responsible for organ development from the endoderm, which in the vertebrate embryo gives rise to the epithelial cells of the alimentary canal, liver, pancreas, lung and thyroid gland.

Pancreas transplantations efficiently restore normoglycemia but requires life-long immunosuppressive therapy and are limited by tissue supply. Differentiated islet cell transplants have a finite lifespan because of inadequate cell renewal. Pancreatic stem cells would have to be used in transplantations to establish a permanent graft but there is not a ready supply of such stem cells.

Ferber et al., (2000) Nature Medicine vol. 6, no. 5 describes the infection of cells with adenovirus encoding the transcription factor PDX-1 which is known to be involved in regulating pancreatic development. It is also known as an insulin transcription factor and is required in differentiated beta cells to activate insulin production. The authors delivered PDX-1 in an adenoviral vector to 11-14 week old mice and showed ectopic expression of PDX-1 mainly in the liver and that this induced expression of the endogenous insulin 1 and 2 genes in liver. The level of PDX-1 produced by transfection was low, with only 0.1 to 1.0% of transfected cells producing insulin. The method described in Ferber et al., does not provide an effective therapy for pancreatic disorders as activation of insulin genes alone would not enable glucose responsiveness suitable for diabetic therapy.

It is an aim of a preferred embodiment of the present invention to provide methods for converting cells of one tissue type into pancreatic cells and materials for use therein, which methods and materials may be used in the treatment of pancreatic disorders such as diabetes and pancreatic cancer.

Accordingly, the present invention in a first aspect provides a method for converting non-pancreatic cells into pancreatic cells, the method comprising providing to the non-pancreatic cells a transcription factor specific for pancreatic cells, in the presence of an activating means able to activate the transcription factor, such that the cells in which the transcription factor is expressed convert into pancreatic cells.

The transcription factor may be provided directly to the cells as a protein. Alternatively or additionally it may be provided by ectopically expressing the transcription factor in the cells.

States of terminal cell differentiation are often considered fixed but in some cases they can inter-convert. The conversion of cells, including stem cells, in postnatal life from one cell type to another is termed metaplasia. The conversion of one cell type to another usually arises in situations of chronic tissue damage and associated regeneration. Some changes may be indirect, occurring through an intervening stem cell, whereas others may be direct transformations, sometimes called trans-differentiations. Numerous examples of metaplasia between endoderm-derived tissues have been described, for example Shen et al., (2000), Nature Cell Biol. Vol. 2 879-887 in relation of the conversion of pancreatic cells into hepatocytes. However, little is known of the molecular and cellular mechanisms involved.

A definition of a “stem cell” is provided by Potten & Loeffler, Development, 110:1001 (1990), who have defined stem cells as “undifferentiated cells capable of (a) proliferation, b) self-maintenance, (c) the production of a large number of differentiated functional progeny, (d) regenerating the tissue after injury, and (e) a flexibility in the use of these options.” Stem cells are used in a body to replace cells that are lost by natural cell death, injury or disease. The presence of stem cells in a particular type of tissue usually correlates with tissues that have a high turnover of cells. Stem cells are also present in tissues, e.g., liver (Travis, Science, 259:1829, 1993), that do not have a high turnover of cells. A “progenitor” cell is typically defined as having the capability to divide for several generations, but not self renew. Progenitor cells also typically are defined to be capable of differentiating into a variety of different cell types.

The inventors propose that by ectopically providing or expressing an active transcription factor specific for pancreatic cells, the cells in which the transcription factor is expressed or to which the transcription factor is provided actually convert into differentiated pancreatic cells or pancreatic stem cells, which stem cells are capable of differentiating into any pancreatic cells to produce pancreatic tissue of equivalent tissue function to that produced naturally. Thus they can convert cells of, for example, a portion of the liver, into functioning pancreatic tissue, containing both endocrine and exocrine cells.

The conversion of tissues into pancreatic tissue by providing or expressing an active transcription factor specific for pancreas is not contemplated in the prior art. Ferber et al., supra suggest that expression of PDX-1 may promote expression of other pancreatic beta cell transcription factors in liver and this may lead to a shift in phenotype of transfected liver cells to a beta cell phenotype. However Ferber goes on to say that, in culture, mature hepatocytes infected with their construct, do not produce insulin, indicating that mature liver cells (in vitro sample) do not convert to a beta cell phenotype, although it is proposed that this may be possible in a pluripotent population of progenitor (undifferentiated) liver cells (in vivo). There is no suggestion that expression of PDX-1 in the liver would convert the treated cells into pancreatic tissue (with both endocrine and exocrine cells) but rather that PDX-1 expression could induce beta cell phenotype in undifferentiated liver cells. Indeed, the expression of PDX-1 in Ferber et al., supra show that the insulin gene can be activated to a low level in liver by introduction of unmodified Pdx1. However, there is no evidence that liver cells are converted to any pancreatic cell type, and in particular no evidence that the full range of pancreatic cell types can be produced. Furthermore, Ferber et al. go on to say that, in culture, mature hepatocytes infected with their construct do not produce insulin.

Transcription factors are proteins that have DNA binding domains capable of binding to specific DNA sequence elements or recognition sites. The binding of transcription factors to such DNA elements (i.e., motifs) in the promoter region of a gene results in the turning on of transcriptional activity, leading to the generation of a messenger RNA and, subsequently, the production of the protein encoded by the gene in the cell. This process is collectively described as gene expression. Transcription factors may be active on their own or act in concert with other proteins, transcriptional modulators, or co-factors to activate transcription. Transcriptional modulators can also act to inhibit the activity of transcription factors to down-regulate gene expression.

Thus, whether a target gene is expressed, and to what extent it is expressed, is regulated at two levels: (1) by the amount of a specific transcription factor produced by or otherwise present in a cell; and (2) by the activity of the transcription factor expressed. These two levels are in turn controlled at the level of cellular signalling, wherein the level of transcription factors produced is influenced by the metabolic or proliferative demands placed on the cell by the hormonal milieu (e.g., in response to insulin, growth factors, see e.g., Calkhoven, C. F., et al., Biochem. J. 317:329-342 (1996)), or by the presence of specific protein partners or cofactors.

As used herein, the term “transcription factor” refers to a protein with a DNA binding domain capable of recognizing and binding to a specific DNA and interacting with a transcription complex. An example of such a transcription factor is PDX-1. The transcription factor coding sequence of the DNA segment can be the same or substantially the same as the coding sequence of the endogenous transcription factor coding sequence as long as it encodes a functional transcription factor protein. Indeed, the DNA segment can also be the same or substantially the same as the transcription factor gene of a non-human species as long as it encodes a functional transcription factor protein. The transcription of the transcription factor gene in the DNA segment is preferably under the control of a promoter sequence different from the promoter sequence controlling the transcription of the endogenous coding sequence.

Genes for transcription factors specific for pancreatic cells are present in all tissues but are only expressed endogenously to any significant degree in pancreatic cells. Transcription factors require protein-protein interactions and an open chromatin state to allow RNA polymerase to function properly to allow gene expression. The inventors propose that proteins specific for pancreatic cells do not function in cells outside the pancreas because the necessary cofactors are not present in these cells to allow transcription. This would explain why the effects of ectopic expression of PDX-1 shown in Ferber et al., supra are so minimal and do not produce ectopic pancreas.

Preferably the non-pancreatic cells are differentiated cells that constitute part of a tissue or organ. Preferably the non-pancreatic cells constitute part of an endodermal organ, for example liver, thyroid, lung or intestine.

To enable sufficient activity of the pancreatic transcription factor in treated cells to allow conversion of treated cells into pancreatic cells an activating means must be introduced into the cells. This activating means may take the form of an activating domain introduced into the cells as nucleic acid by any suitable means, for example transfection of the cells with a nucleic acid construct. The activating means may also take the form of some other upstream substance or treatment that is able to active a pancreatic transcription factor gene not normally expressed in non-pancreatic cells.

The activating means may take the form of an activating protein introduced into the cells by any suitable means. Preferably, the activating means in a nucleic acid.

The inventors have found that the transcriptional activation domain VP16 (Sadowski et al., (1988) Nature 335, 5634) from Herpes simplex virus and functional homologues thereof, is particularly effective in allowing a high level of activity of pancreas-specific transcription factors specific for pancreatic cells in cells other than pancreatic cells. It is also proposed that when cDNA encoding VP16 or its functional homologues are fused to the cDNA of the appropriate pancreatic transcription factors, the resultant fusion protein would then be able to induce expression of endogenous genes specific for pancreatic cells in cells other than pancreatic cells. VP16 is known to function as a strong transactivator and to open up chromatin and has been used previously in Xenopus to determine whether certain transcription factors function as activators or repressors.

VP16 was originally called VMW65 and is an “immediate early” protein of Herpes simplex virus. Generally a subfragment of VP16 comprising the C-termianl 78 amino acids is used as a transcriptional activation domain.

Introduction of the activation means may bring about expression of an endogenous pancreatic transcription factor in non-pancreatic cells or influence the expression or activity of an exogenous pancreatic transcription factor introduced into the cell by any suitable means.

The inventors propose that in either case, once the endogenous or exogenous transcription factor has been expressed ectopically or otherwise provided to the cells, the cells will convert to pancreatic cells, in which case the endogenous pancreatic transcription factors will be naturally expressed.

Preferably the activation means comprises a genetically introduced transcription activation domain. Such domains according to the present invention may be selected from peptide sequences of naturally occurring transcription factors such as the widely used transcription activation domain of Herpes Simplex Virus VP16, may be derived from such sequences or may comprise a composite transcription activation region. A composite transcription activation region consists of a continuous polypeptide region containing two or more reiterated or mutually heterologous component polypeptide portions. The component polypeptide portions comprise polypeptide sequences derived from at least two different proteins, polypeptide sequences from at least two non-adjacent portions of the same protein, polypeptide sequences which are not found so linked in nature (including reiterated copies of a polypeptide sequence) or non-naturally occurring peptide sequence.

Transcription factors of this invention may contain, in addition to one or more copies of a primary activation domain such as described above, one or more copies of one or more heterologous peptide sequences which potentiate the transcription activation potency of the transcription factor, as measured by any means. Inclusion of such motifs, including the so-called “glutamine-rich”, “proline-rich” and “acidic” transcription activation motifs, in combination with a primary activation domain can result in extremely high levels of transcription.

Preferably, the method according to the first aspect of the invention comprises introducing into non-pancreatic cells isolated nucleic acid encoding activating means fused to isolated nucleic acid encoding a pancreatic transcription factor.

In one embodiment, nucleic acid is introduced into cells in vitro, which cells are introduced into a patient, and, as a result, the cells express in vivo in a patient a therapeutically effective amount of transcription factor so as to produce additional pancreatic tissue.

In another embodiment, the nucleic acid is directly introduced to a patient in vivo, e.g., not contained within a cell. The nucleic acid can be introduced in vivo in a vector. Examples of suitable vectors include viral vectors (e.g., retroviral vectors, adenoviral vectors, adeno-associated viral vectors and herpes viral vectors), plasmids, cosmids, and yeast artificial chromosomes. The nucleic acid can also be introduced as infectious particles, e.g., DNA-ligand conjugates, calcium phosphate precipitates, and liposomes.

Numerous techniques are known in the art for the introduction of foreign genes into cells and these include transformation, transfection, homologous recombination and electroporation (see for example Loeffler and Behr, 1993, Meth. Enzymol. 217: 599-618; Cohen et al., 1993, Meth. Enzymol. 217: 618-644; Cline, 1985, Pharmac. Ther. 29:69-92).

Preferably the pancreatic transcription factor is targeted to cells of a particular tissue. Preferably the pancreatic transcription factor is introduced into the cells of a particular tissue under the control of a tissue specific promoter to allow targeted expression of the transcription factor in that particular tissue.

For example, the pancreatic transcription factor may be expressed in liver cells under the control of a liver specific promoter, such as the transthyretin (TTR), glucose 6 phosphatase or albumin promoters. The pancreatic transcription factor may be expressed in intestinal cells under the control of a promoter specific to intestinal cells, such as the intestinal fatty acid binding protein (IFABP) promoter. The pancreatic transcription factor specific for pancreatic cells may be expressed in lung cells under the control of a promoter specific to lung cells such as the surfactant A or surfactant C promoter. The pancreatic transcription factor specific for pancreatic cells may be expressed in thyroid cells under the control of a promoter specific to thyroid cells such as the thyroglobulin promoter.

In a preferred embodiment of the present invention, the means for expressing a transcription factor for pancreatic cells in non-pancreatic cells comprises transfection of the non-pancreatic cells with a nucleic acid construct comprising an activating domain and an isolated pancreatic transcription factor gene, whose expression is under the control of a tissue specific promoter.

The term “gene” as described herein is intended to include cDNA encoding the full length pancreatic transcription factor as well as functional subfragments and variants thereof, including insertions, additions and deletions which encode a functioning pancreatic transcription factor. Genes (cDNA) having extensions at the 5′ or 3′ are also encompassed within the scope of the present invention.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52: 456 (1973); Sambrook et al., Molecular Cloning, a laboratory Manual, Cold Spring Harbor Laboratories (New York, 1989); Davis et al., Basic Methods in Molecular Biology, Elsevier, 1986; and Chu et al., Gene, 13: 197 (1981). Such techniques can be used to introduce one or more exogenous DNA moieties into suitable host cells.

The cells containing the nucleic acid construct mentioned above are prepared by introduction of the desired DNA constructs, linked or unlinked to each other, using any methods and materials permitting introduction of heterologous DNA into cells. For instance, the constructs may be introduced into the cell by calcium phosphate precipitation, DEAE dextran-DNA complexation, fusion, electroporation, biolistics, transfection, lipofection etc. Various types of DNA vectors are known which may be used, including retroviral, adenoviral, adenoassociated viral, BPV, etc. The engineered cells may be cultured and the introduced DNA may be permitted to integrate into the host cell's chromosomal material, although integration is not necessary since transient production of protein is likely to be sufficient in order to switch cells permanently to a pancreatic phenotype. The engineered cells may be characterized as desired and may be encapsulated within a variety of semipermeable materials prior to introduction into the host organism using known methods.

As an alternative to the introduction of genetically engineered cells into the whole organism, the various DNA constructs may be introduced directly into the host organism using materials, methods and conditions permitting DNA uptake by one or more cells within the organism, e.g. using direct injection, liposomes, or DNA vectors including viral vectors such as retroviral vectors, adenoviral vectors, or adeno-associated (AAV) vectors.

Alternatively, either one or both of the transcription factor and activating means may be provided to a cell by the introduction of RNA or protein. RNA and protein-based approaches may be advantageous because unlike the case with approaches that introduce DNA, there is no risk of genomic damage or changes to the genetic makeup of germline cells.

Preferably the pancreatic transcription factor ectopically expressed or otherwise provided to a cell according to the present invention is the pancreatic transcription factor PDX-1 (mouse) or homologues thereof, such as X1Hbox8 (xenopus), STF-1 (rat) or IPF-1 (human). References to PDX-1 include Wright et al., (1988) Development 104, 787-794 (Xenopus) and Ohlsson et al., (1993) EMBO J. 12, 4251-4259 (mammal). Other preferred pancreatic transcription factors include neurogenin 3 and p48 (see Example 5 for further details). For a recent review of pancreatic transcription factors that may be use in accordance with the methods of the present invention see Wilson, M E et al., (2003) “Gene expression cascades in pancreatic development” Mech. Dev. 120, 65-80.

The method contemplates either turning on expression and activity of an endogenous PDX-1 gene or introduction of an exogenous PDX-1 gene (as DNA or RNA) which is capable of being expressed as an active transcription factor in the cell into which is has been introduced, or introduction of exogenous PDX-1 protein into a cell. Nucleic acids which encode derivatives (including fragments) or analogues of PDX-1 and homologues can also be used according to the present invention, as long as the derivatives and analogues retain their ability to provide pancreatic transcription factor function. Similarly, derivatives, analogues and homologues of PDX protein can be used. Persistence of the introduced transcription factor is not necessarily required; because when the cells have switched to a pancreatic type their differentiation state is permanent.

The method according to the present invention may be used to generate pancreatic tissue in patients, for example to treat those suffering from loss of pancreatic tissue or function thereof, such as those patients suffering from diabetes or pancreatic cancer. The method according to the present invention may be used as a long term treatment or even cure for diabetes, to be achieved by converting part of the liver, or other tissues, to functioning pancreas tissue. The method according to the present invention is especially applicable to cases of diabetes where the existing treatment comprises insulin supplementation, that is, all type 1 diabetes and some cases of type 2 diabetes. Delivery of the nucleic acid or protein into a patient may be either direct, in which case the patient is directly exposed to the nucleic acid, a nucleic acid carrying vector or the protein, or indirect, in which case cells are first transformed with nucleic acid or provided with protein in vitro, then implanted into the patient. These cells may be differentiated cells or stem cells and may originate from the patient into which they are implanted (reimplantation) or from elsewhere (transplantation). These two approaches are known respectively as in vivo or ex vivo gene therapy when used with nucleic acid.

There may be advantages in treated a patient with cells derived from the patient's own body because the risk of transmission of an infection such as HIV is eliminated and the risk of triggering an immune system-mediated rejection reaction is reduced.

Regardless of whether in vivo or ex vivo gene therapy is used, the necessary reagents or cells may be introduced into the patient by any suitable route whether that route be enteral or parenteral, for example, intravenous or intramuscular. It may be preferable to introduce reagents or cells directly into the site at which they will subsequently remain.

For treatment in vivo, nucleic acid constructs comprising a pancreatic transcription factor (e.g. PDX-1) and an activating domain (e.g. VP16) may be injected intravenously into the bloodstream of a patient in a dose dependent manner so as to convert part of a tissue into pancreatic tissue.

For treatment ex vivo, which is especially effective for liver cells, autologous cells may be cultured and transfected with a nucleic acid construct comprising a pancreatic transcription factor (e.g. PDX-1) and an activating domain (e.g. VP16). Transfected cells form a localised mass of pancreatic tissue which may be reintroduced into the patient.

When cells are transformed with the nucleic acid for a pancreatic transcription factor in vitro it is helpful to provide means of monitoring the success of any transfection step. Accordingly, it is preferred that a nucleic acid construct used for transfecting non-pancreatic cells contains a reporter gene. The reporter gene preferably comprises a light emitting reporter gene, for example one that encodes a protein that is fluorescent. A preferred reporter gene is green fluorescent protein (GFP) and light emitting derivatives thereof. GFP is from the jelly fish Aquorea victoria and is able to absorb blue light and re-emits an easily detectable green light. GFP may be advantageously used as a reporter because its measurement is simple and reagent free and the protein is non-toxic. Generally the GFP reporter gene is used in nucleic acid constructs under the control of an elastase promoter (nucleotides −205 to +8), which is capable of driving expression in both endocrine and exocrine cells (Kruse et al., (1993) Genes Dev. 7. 774-786). If GFP has been transfected into cells, clusters of green fluorescence may be observed.

According to the present invention in a second aspect there is provided a nucleic acid construct comprising a pancreatic transcription factor and an activating domain that is capable of activating the transcription factor in cells into which the nucleic acid construct is introduced. Preferably the cells into which the nucleic acid construct is introduced are non-pancreatic cells, more preferably, liver cells.

The nucleic acid construct may be used according to the method of the first aspect of the invention to convert non-pancreatic cells into pancreatic tissue.

The pancreatic transcription factor preferably comprises PDX-1 (mouse) or homologues thereof, such as X1Hbox8 (Xenopus), STF-1 (rat) or IPF-1 (human) or neurogenin 3 or homologues thereof, or p48 or homologues thereof. Nucleic acids which encode derivatives (including fragments) or analogues of PDX-1, neurogenin 3 or P48 or homologues of any thereof can also be used in the construct according to the present invention, as long as the derivatives and analogues retain their ability to provide pancreatic transcription factor function. Preferably, the transcription factor is PDX-1 or a homologue thereof.

It may be possible to use a transcription factor that is from the same species as the cells to be transdifferentiated. For example X1Hbox8, the Xenopus homologue of PDX-1, may be used for transdifferentiation of Xenopus cells or human cells. In fact X1Hbox8 in human cells shows greater activity than PDX-1 in human cells and may therefore be preferred to PDX-1 for use in human cells under some circumstances.

The activating domain preferably comprises the transcriptional activation domain VP16 from Herpes simplex virus and functional homologues, analogues and derivatives thereof which maintain VP16 activity in activating RNA polymerase.

Preferably the nucleic acid construct further comprises a promoter. The promoter may be constitutive or inducible. Preferably the promoter is tissue specific, for example a liver specific promoter such as the transthyretin (TTR), glucose 6 phosphatase or albumin promoter, an intestinal cell specific promoter, such as the intestinal fatty acid binding protein (IFABP) promoter, a thyroid specific promoter, such as the thyroglobulin promoter, or a lung cell specific promoter such as the surfactant A or surfactant C promoter.

To allow the success of transformation experiments to be monitored, the nucleic acid construct according to the present invention preferably further comprises a reporter gene. A preferred reporter gene is green fluorescent protein (GFP). Generally the GFP reporter gene is used in nucleic acid constructs under the control of the elastase promoter, which is capable of driving expression in both endocrine and exocrine cells. If GFP has been transfected into cells, clusters of green fluorescence may be observed. Promoters other than elastase, which drive expression in specific types of pancreatic cells may also be used according to the present invention, for example to study expression in D cells or duct cells.

A nucleic acid construct provided as a preferred embodiment of the present invention, for use in Xenopus, comprises the TTR promoter operably linked to the X1Hbox8 gene and VP16, linked to the elastase promoter controlling expression of GFP.

Vectors comprising the nucleic acid construct according to the second aspect of the invention, and non-pancreatic cells transfected with such vectors are also contemplated as third and fourth aspects of the invention.

A fifth aspect of the invention provides compositions comprising a component for providing to a cell a transcription factor specific for pancreatic cells and a component comprising an activating means, wherein at least one of the above components is provided as a protein and the other component is provided as a protein or nucleic acid.

Prefered features of the protein transcription factor or activatory means that may be present in the composition according to the fifth aspect of the invention are mutatis mutandis as described above in relation to the first aspect of the invention.

A sixth aspect of the invention provides pancreatic tissue of non pancreatic origin produced by the method of the first aspect of the invention or by transformation of non-pancreatic cells with the nucleic acid construct according to the second aspect of the invention or by treatment of non-pancreatic cells with the composition of the fifth aspect of the invention.

The seventh aspect of the invention provides a method of treatment of pancreatic disorders comprising converting non-pancreatic cells into pancreatic tissue according to the method of the first aspect of the invention or by transformation of non-pancreatic cells with the nucleic acid construct according to the second aspect of the invention or by treatment of non-pancreatic cells with the composition of the fifth aspect of the invention.

Preferably the pancreatic disorders that may be treated according to the seventh aspect of the invention include diabetes and pancreatic cancer.

The present invention in a eighth aspect provides for the use of the nucleic acid construct according to the second aspect of the invention, or the vector according to the third aspect of the invention, or the cells according to the fourth aspect of the invention, or the compositions according to the fifth aspect of the invention, or the pancreatic tissue according to the sixth aspect of the invention in therapy.

The present invention in a ninth aspect provides for the use of the nucleic acid construct according to the second aspect of the invention, or the vector according to the third aspect of the invention, or the cells according to the fourth aspect of the invention, or the compositions according to the fifth aspect of the invention, or the pancreatic tissue according to the sixth aspect of the invention in the manufacture of a medicament for pancreatic disorders such as diabetes or pancreatic cancer.

The present invention in a tenth aspect provides the nucleic acid construct according to the second aspect of the invention, or the vector according to the third aspect of the invention, or the cells according to the fourth aspect of the invention, or the compositions according to the fifth aspect of the invention, or the pancreatic tissue according to the sixth aspect of the invention for use as a medicament

Definitions

“Nucleic acid” as used herein refers to an oligonucleotide, nucleotide, and fragments or portions thereof, as well as to peptide nucleic acids (PNA), fragments, portions or antisense molecules thereof, and to DNA or RNA of genomic or synthetic origin which can be single- or double-stranded, and represent the sense or antisense strand. Where “nucleic acid” is used to refer to a specific nucleic acid sequence “nucleic acid” is meant to encompass polynucleotides that encode a polypeptide that is functionally equivalent to the recited polypeptide, e.g., polynucleotides that are degenerate variants, or polynucleotides that encode biologically active variants or fragments of the recited polypeptide, including polynucleotides having substantial sequence similarity or sequence identity relative to the sequences provided herein.

Similarly, “polypeptide” and “protein” as used herein refers to an oligopeptide, peptide, or protein. Where “polypeptide” or “protein” are recited herein to refer to an amino acid sequence of a naturally-occurring protein molecule, “polypeptide” or “protein” are not meant to limit the amino acid sequence to the complete, native amino acid sequence associated with the recited protein molecule, but instead is meant to also encompass biologically active variants or fragments, including polypeptides having substantial sequence similarity or sequence identify relative to the amino acid sequences provided herein.

As used herein, “polypeptide” and “protein” refer to an amino acid sequence of a recombinant or non-recombinant polypeptide having an amino acid sequence of i) a native polypeptide, ii) a biologically active fragment of an polypeptide, iii) biologically active polypeptide analogues of an polypeptide, or iv) a biologically active variant of an polypeptide.

Polypeptides and protein useful in the invention can be obtained from any species, e.g., mammalian or non-mammalian (e.g., reptiles, amphibians, avian (e.g., chicken)), particularly mammalian, including human, rodent (e.g., murine or rat), bovine, ovine, porcine, murine, or equine, preferably rat or human, from any source whether natural, synthetic, semi-synthetic or recombinant.

A “variant” of a polypeptide or protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological or immunological activity can be found using computer programs well known in the art.

A “deletion” is defined as a change in either amino acid or nucleotide sequence in which one or more amino acid or nucleotide residues, respectively, are absent as compared to an amino acid sequence or nucleotide sequence of a naturally occurring polypeptide or protein of interest.

An “insertion” or “addition” is that change in an amino acid or nucleotide sequence which has resulted in the addition of one or more amino acid or nucleotide residues, respectively, as compared to an amino acid sequence or nucleotide sequence of a naturally occurring polypeptide.

A “substitution” results from the replacement of one or more amino acids or nucleotides by different amino acids or nucleotides, respectively as compared to an amino acid sequence or nucleotide sequence of a naturally occurring polypeptide.

The term “derivative” as used herein refers to the chemical modification of a nucleic acid encoding a polypeptide or the encoded polypeptide. Illustrative of such modifications would be replacement of hydrogen by an alkyl, acyl, or amino group. A nucleic acid derivative would encode a polypeptide which retains essential biological characteristics of a natural polypeptide.

As used herein the term “isolated” is meant to describe a compound of interest (e.g., either a polynucleotide or a polypeptide) that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound (e.g., either a polynucleotide or a polypeptide) that is removed from its natural environment and is at least 60% free, preferably 75% free, and most preferably 90% free from other components with which it is naturally associated.

By “transformation” or “transfection” is meant a permanent or transient genetic change, preferably a permanent genetic change, induced in a cell following incorporation of new nucleic acid (e.g., DNA or RNA exogenous to the cell). Genetic change can be accomplished either by incorporation of the new nucleic acid into the genome of the host cell, or by transient or stable maintenance of the new DNA as an episomal element.

By “transformed cell” is meant a cell into which (or into an ancestor of which) has been introduced, by means of recombinant DNA techniques, a DNA molecule encoding a protein of interest.

By “construct” is meant a recombinant nucleic acid, generally recombinant DNA, that has been generated for the purpose of the expression of a specific nucleotide sequence(s), or is to be used in the construction of other recombinant nucleotide sequences.

By “vector” is meant any compound, biological or chemical, which facilitates transformation of a target cell with a DNA of interest. Exemplary biological vectors include viruses, particularly attenuated and/or replication-deficient viruses. Exemplary chemical vectors include lipid complexes and naked DNA constructs.

By “naked DNA” or “naked nucleic acid” or DNA sequence and the like is meant a nucleic acid molecule that is not contained within a viral particle, bacterial cell or other encapsulating means that facilitates delivery of nucleic acid into the cytoplasm of the target cell. Naked nucleic acid can optionally be associated (e.g. formulated) with means for facilitating delivery of the nucleic acid to the site of the target cell (e.g., means that facilitate travel into the cell, protect the nucleic acid from nuclease degradation, and the like) and/or to the surface of the target epithelial cell.

By “promoter” is meant a minimal sequence sufficient to direct transcription. “Promoter” is also meant to encompass those promoter elements sufficient for promoter-dependent gene expression controllable for cell-type specific, tissue-specific or inducible by external signals or agents; such elements may be located in the 5′ or 3′ regions of the native gene.

By “operably linked” or “operatively linked” is meant that a DNA sequence and a regulatory sequence(s) are connected in such a way as to permit expression when the appropriate molecules (e.g., transcriptional activator proteins) are bound to the regulatory sequence(s).

The term “pancreatic transcription factor gene” is used to designate both transcription factors that are expressed in pancreatic cells, and also transcription factors that are involved in the development, differentiation, or formation of pancreatic cells. The term “pancreatic transcription factor gene” is also intended to mean the open reading frame encoding specific pancreatic transcription factor polypeptides, introns, and adjacent 5′ and 3′ non-coding nucleotide sequences involved in the regulation of expression, up to about 10 kb beyond the coding region, but possibly further in either direction. The DNA sequences encoding a pancreatic transcription factor may be cDNA or genomic DNA or a fragment thereof. The gene may be introduced into an appropriate vector for extrachromosomal maintenance or for integration into the host.

The term “cDNA” as used herein is intended to include all nucleic acids that share the arrangement of sequence elements found in native mature mRNA species, where sequence elements are exons (e.g., sequences encoding open reading frames of the encoded polypeptide) and 3′ and 5′ non-coding regions. Normally mRNA species have contiguous exons, with the intervening introns removed by nuclear RNA splicing, to create a continuous open reading frame encoding the polypeptide of interest.

The term “ectopic” as used herein is intended to include the expression, activity, provision or presence of a nucleic acid, protein, composition or other substance at a body site, tissue, organ or cell type at which it is not normally to be found or at which it would not otherwise be found in the individual animal concerned. The term also includes the presence of a cell at a body site, tissue or organ at which it is not normally to be found or at which it would not otherwise be found in the individual animal concerned.

A pancreatic transcription factor genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a large fragment of 100 kbp or more, or as a smaller fragment substantially free of flanking chromosomal sequence.

The sequence of this 5′ region, and further 5′ upstream sequences and 3′ downstream sequences, may be utilized for promoter elements, including enhancer binding sites, that provide for expression in tissues where the islet transcription factor is expressed. The sequences of the pancreatic transcription factor promoter elements of the invention can be based on the nucleotide sequences of any species, either vertebrate or invertebrate and can be isolated or produced from any source whether natural, synthetic, semi-synthetic or recombinant.

The nucleic acid compositions used in the subject invention may encode all or a part of the pancreatic transcription factor polypeptides as appropriate. Fragments may be obtained of the DNA sequence by chemically synthesizing oligonucleotides in accordance with conventional methods, by restriction enzyme digestion, by PCR amplification, etc. For the most part, DNA fragments will be of at least about ten contiguous nucleotides, usually at least about 15 nucleotides (nt), more usually at least about 18 nt to about 20 nt, more usually at least about 25 nt to about 50 nt. Such small DNA fragments are useful as primers for PCR, hybridization screening, etc. Larger DNA fragments, i.e. greater than 100 nt are useful for production of the encoded polypeptide. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The pancreatic transcription factor genes are isolated and obtained in substantial purity, generally as other than an intact mammalian chromosome. Usually, the DNA will be obtained substantially free of other nucleic acid sequences that do not include a sequence encoding an islet transcription factor or fragment thereof generally being at least about 50%, usually at least about 90% pure and are typically “recombinant”, i.e. flanked by one or more nucleotides with which it is not normally associated on a naturally occurring chromosome.

The sequence of a pancreatic transcription factor, including flanking promoter regions and coding regions, may be mutated in various ways known in the art to generate targeted changes in promoter strength, sequence of the encoded protein, etc. The DNA sequence or product of such a mutation will be substantially similar to the sequences provided herein, i.e. will differ by at least one nucleotide or amino acid, respectively, and may differ by at least two, or by at least about ten or more nucleotides or amino acids. In general, the sequence changes may be substitutions, insertions or deletions. Deletions may further include larger changes, such as deletions of a domain or exon. Such modified islet transcription factor sequences can be used, for example, to generate vectors for introduction into target cells for the purpose of producing islet cells.

Techniques for in vitro mutagenesis of cloned genes are known. Examples of protocols for scanning mutations may be found in Gustin et al., 1993 Biotechniques 14:22; Barany, 1985 Gene 37:111-23; Colicelli et al., 1985 Mol Gen Genet 199:537-9; and Prentki et al., 1984 Gene 29:303-13. Methods for site specific mutagenesis can be found in Sambrook et al., 1989 Molecular Cloning: A Laboratory Manual, CSH Press, pp. 15.3-15.108; Weiner et al., 1993 Gene 126:35-41; Sayers et al., 1992 Biotechniques 13:592-6; Jones and Winistorfer, 1992 Biotechniques 12:528-30; Barton et al., 1990 Nucleic Acids Res 18:7349-55; Marotti and Tornich, 1989 Gene Anal Tech 6:67-70; and Zhu 1989 Anal Biochem 177: 120-4.

A pancreatic transcription factor of particular interest in the present invention is a member of the homeobox-type group of transcription factor proteins, e.g., PDX-1. References to PDX-1 include Wright et al., (1988) Development 104, 787-794 (Xenopus) and Ohlsson et al., (1993) EMBO J. 12, 4251-4259 (mammal). References to neurogenin 3 and p48 include Wilson et al, (2003) Mechanisms of Development 120,65-80.

It should be noted that transcription factors which act either “upstream” of a pancreatic transcription factor (and therefore activate the transcription factor) or “downstream” of a pancreatic transcription factor, that lead to development of the pancreatic cell phenotype, are also contemplated for use in the present invention.

Where the pancreatic transcription factor nucleic acid to be delivered is DNA, any construct having a promoter (e.g., a promoter that is functional in a eukaryotic cell) operably linked to a DNA of interest can be used in the invention. The constructs containing the DNA sequence (or the corresponding RNA sequence) that may be used in accordance with the invention may be any eukaryotic expression construct containing the DNA or the RNA sequence of interest. For example, a plasmid or viral construct (e.g. adenovirus) can be cleaved to provide linear DNA having ligatable termini. These termini are bound to exogenous DNA having complementary-like ligatable termini to provide a biologically functional recombinant DNA molecule having an intact replicon and a desired phenotypic property. Preferably the construct is capable of replication in eukaryotic and/or prokaryotic hosts (viruses in eukaryotic, plasmids in prokaryotic), which constructs are known in the art and are commercially available.

The constructs can be prepared using techniques well known in the art. Likewise, techniques for obtaining expression of exogenous DNA or RNA sequences in a genetically altered host cell are known in the art (see, for example, Kormal et al., Proc. Natl. Acad. Sci. USA, 84:2150-2154, 1987; Sambrook et al. Molecular Cloning: a Laboratory Manual, 2nd Ed., 1989, Cold Spring Harbor Laboratory Press, Cold Spring Harbor, N.Y.).

In one embodiment, the DNA construct contains a promoter to facilitate expression of the DNA of interest within a non-pancreatic cell. The promoter may be a strong, viral promoter that functions in eukaryotic cells such as a promoter from cytomegalovirus (CMV), mouse mammary tumor virus (MMTV), Rous sarcoma virus (RSV), or adenovirus. More specifically, exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982). Of these two promoters, the CMV promoter is presently preferred as it provides for higher levels of expression than the RSV promoter.

Alternatively, the promoter used may be a strong general eukaryotic promoter such as the actin gene promoter.

Tissue specific promoters are particularly preferred, especially the TTR, liver specific promoter (Yan, C et al., (1999) EMBO J. 9, 869-878). The constructs of the invention may also include sequences in addition to promoters which enhance expression in the target cells.

In another embodiment, the promoter is a regulated promoter, such as a tetracycline-regulated promoter, expression from which can be regulated by exposure to an exogenous substance (e.g., tetracycline.).

Other components such as a marker (e.g., an antibiotic resistance gene (such as an ampicillin resistance gene) or beta-galactosidase) aid in selection or identification of cells containing and/or expressing the construct, an origin of replication for stable replication of the construct in a bacterial cell (preferably, a high copy number origin of replication), a nuclear localization signal, or other elements which facilitate production of the DNA construct, the protein encoded thereby, or both.

For eukaryotic expression, the construct should contain at a minimum a eukaryotic promoter operably linked to a DNA of interest, which is in turn operably linked to a polyadenylation signal sequence. The polyadenylation signal sequence may be selected from any of a variety of polyadenylation signal sequences known in the art. An exemplary polyadenylation signal sequence is the SV40 early polyadenylation signal sequence. The construct may also include one or more introns, where appropriate, which can increase levels of expression of the DNA of interest, particularly where the DNA of interest is a cDNA (e.g., contains no introns of the naturally-occurring sequence). Any of a variety of introns known in the art may be used (e.g., the human beta-globin intron, which is inserted in the construct at a position 5′ to the DNA of interest).

In an alternative embodiment, the nucleic acid delivered to the cell is an RNA encoding a pancreatic transcription factor. In this embodiment, the RNA is adapted for expression (i.e., translation of the RNA) in a target cell. Methods for production of RNA (e.g., mRNA) encoding a protein of interest are well known in the art, and can be readily applied to the product of RNA encoding pancreatic transcription factors useful in the present invention.

Delivery of pancreatic transcription factor-encoding nucleic acid can be accomplished using a viral or a non-viral vector. In one embodiment the nucleic acid is delivered within a viral particle, such as an adenovirus. In another embodiment, the nucleic acid is delivered in a formulation comprising naked DNA admixed with an adjuvant such as viral particles (e.g., adenovirus) or cationic lipids or liposomes. An “adjuvant” is a substance that does not by itself produce the desired effect, but acts to enhance or otherwise improve the action of the active compound. The precise vector and vector formulation used will depend upon several factors, such as the size of the DNA to be transferred, the delivery protocol to be used, and the like.

In general, viral vectors used in accordance with the invention are composed of a viral particle derived from a naturally-occurring virus which has been genetically altered to render the virus replication-defective and to deliver a recombinant gene of interest for expression in a target cell in accordance with the invention.

Numerous viral vectors are well known in the art, including, for example, retrovirus, adenovirus, adeno-associated virus, herpes simplex virus (HSV), cytomegalovirus (CMV), vaccinia and poliovirus vectors. Adenovirus and AAV are usually preferred viral vectors since these viruses efficiently infect slowly replicating and/or terminally differentiated cells. The viral vector may be selected according to its preferential infection of the cells targeted.

Where a replication-deficient virus is used as the viral vector, the production of infectious virus particles containing either DNA or RNA corresponding to the DNA of interest can be achieved by introducing the viral construct into a recombinant cell line which provides the missing components essential for viral replication. In one embodiment, transformation of the recombinant cell line with the recombinant viral vector will not result in production or substantial production of replication-competent viruses, e.g., by homologous recombination of the viral sequences of the recombinant cell line into the introduced viral vector. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus). Methods and materials for manipulation of the mumps virus genome, characterization of mumps virus genes responsible for viral fusion and viral replication, and the structure and sequence of the mumps viral genome are described in Tanabayashi et al., J. Virol. 67:2928-2931, 1993; Takeuchi et al., Archiv. Virol., 128:177-183, 1993; Tanabayashi et al., Virol. 187:801-804, 1992; Kawano et al., Virol., 179:857-861, 1990; Elango et al., J. Gen. Virol. 69:2893-28900, 1988.

The nucleic acid of interest may be introduced into a cell using a non-viral vector. “Non-viral vector” as used herein is meant to include naked DNA (e.g., DNA not contained within a viral particle, and free of a carrier molecules such as lipids), chemical formulations comprising naked nucleic acid (e.g., a formulation of DNA (and/or RNA) and cationic compounds (e.g., dextran sulfate, cationic lipids)), and naked nucleic acid mixed with an adjuvant such as a viral particle (e.g., the DNA of interest is not contained within the viral particle, but the formulation is composed of both naked DNA and viral particles (e.g., adenovirus particles) (see, e.g., Curiel et al. 1992 Am. J. Respir. Cell Mol. Biol. 6:247-52). Thus “non-viral vector” can include vectors composed of nucleic acid plus viral particles where the viral particles do not contain the DNA of interest within the viral genome.

The nucleic acid can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the invention include DOPE (dioleyl phosphatidyl ethanol amine), CUDMEDA (N-(5-cholestrum-3-beta.-ol 3-urethanyl)-N′,N′-dimethylethylene diamine).

For example, the naked DNA can be administered in a solution containing Lipofectin™ (LTI/BRL) at a concentrations ranging from about 2.5% to 15% volume: volume, preferably about 6% to 12% volume:volume.

The nucleic acid of interest can also be administered as a chemical formulation of DNA or RNA coupled to a carrier molecule (e.g., an antibody or a receptor ligand) which facilitates delivery to host cells for the purpose of altering the biological properties of the host cells. By the term “chemical formulations” is meant modifications of nucleic acids which allow coupling of the nucleic acid compounds to a carrier molecule such as a protein or lipid, or derivative thereof. Exemplary protein carrier molecules include antibodies specific to the cells of a targeted pancreatic cell or receptor ligands, e.g., molecules capable of interacting with receptors associated with a cell of a targeted pancreatic cell.

Nucleic acid encoding PDX-1 or other pancreatic transcription factors of interest may be employed to synthesize full-length polypeptides or fragments thereof, particularly fragments corresponding to functional domains; DNA binding sites; etc.; and including fusions of the subject polypeptides to other proteins or parts thereof. Accordingly, the polynucleotides and polypeptides suitable for use in the invention include, without limitation, pancreatic transcription factor polypeptides and polynucleotides found in primates, rodents, canines, felines, equines, nematodes, yeast and the like, and the natural and non-natural variants thereof.

Nucleic acid encoding a pancreatic transcription factor (e.g., PDX-1) can be introduced into a cell in vitro in the presence of a transcription activating means (e.g. VP16) to accomplish ectopic expression of the transcription factor. The cells into which the nucleic acid is introduced can be differentiated epithelial cells (e.g., gut cells, hepatic cells or duct cells), pluripotent adult or embryonic stem cells, or any mammalian cell capable of developing into pancreatic cells or cells capable of functioning as pancreatic tissue in vitro following expression of a pancreatic transcription factor-encoding nucleic acid. The cell is subsequently implanted into a subject having a disorder characterized by a deficiency in insulin, which disorder is amenable to treatment by pancreatic cell replacement therapy. In one embodiment, the host cell in which pancreatic transcription factor (eg, PDX-1) ectopic expression is provided and which is implanted in the subject is derived from the individual who will receive the transplant (e.g., to provide an autologous transplant). For example, in a subject having Type 1 diabetes, pluripotent stem cells, hepatic cells or gut cells can be isolated from the affected subject, the cells modified to express PDX-1-encoding DNA, and the cells implanted in the affected subject to provide pancreatic tissue, or the transformed cells cultured so as to facilitate development of the cells into pancreatic tissue. Alternatively, pluripotent stem cells, hepatic cells or gut cells from another subject (the “donor”) could be modified to express PDX-1-encoding DNA, and the cells subsequently implanted in the affected subject to provide pancreatic tissue, or the transformed cells cultured so as to facilitate development of the cells into pancreatic tissue.

Introduction of nucleic acid into the cell in vitro can be accomplished according to methods well known in the art (e.g., through use of electroporation, microinjection, lipofection infection with a recombinant (preferably replication-deficient) virus, and other means well known in the art). The nucleic acid is generally operably linked to a promoter that facilitates a desired level of polypeptide expression (e.g., a promoter derived from CMV, SV40, adenovirus, or a tissue-specific or cell type-specific promoter). Transformed cells containing the recombinant nucleic acid can be selected and/or enriched via, for example, expression of a selectable marker gene present in the introduced construct or that is present on a nucleic acid that is co-transfected with the construct. Typically selectable markers provide for resistance to antibiotics such as tetracycline, hygromycin, neomycin, and the like. Other markers can include thymidine kinase and the like. Other markers can include markers that can be used to identify expressing cells, such as beta-galactosidase or green fluorescent protein.

Expression of the introduced nucleic acid in the transformed cell can be assessed by various methods known in the art. For example, expression of the introduced gene can be examined by Northern blot to detect mRNA which hybridizes with a DNA probe derived from the relevant gene. Those cells that express the desired gene can be further isolated and expanded in in vitro culture using methods well known in the art. The host cells selected for transformation will vary with the purpose of the ex vivo therapy, the site of implantation of the cells, and other factors that will vary with a variety of factors that will be appreciated by the ordinarily skilled artisan.

Compositions in accordance with the fifth aspect of the invention may be introduced in vitro or in vivo into cells by methods well known in the art (e.g., through use of elctroporation, microinjection, liposomes and biolistics).

It may not be necessary for the transcription factor to be permanently expressed in a cell in order for the cell to become permanently transdifferentiated. This assertion is supported by experimental observations where expression of PDX-1 is undetectable in fully differentiated endogenous pancreas. Although PDX-1 has an important role in initial pancreas differentiation, it is proposed, whilst not wishing to be bound by any particular theory, that once differentiation is complete it is maintained by alternative mechanisms and expression of PDX-1 is either reduced to very low levels or completely abolished. The failure to detect PDX-1 in transdifferentiated tissue suggests that the methods of transdifferention described herein does not necessarily require permanent expression of PDX-1. Consequently the invention includes methods of transdifferentiation wherein the transcription factor specific for pancreatic cells is under the control of a transiently-activated or inducible promoter. Alternatively or additionally, transient activity of a transcription factor for pancreatic cells may be achieved by the use of an RNA construct, which will degrade after a period within a cell or after a certain number of translations or by use of a protein which will degrade after a period of intracellular activity. RNA and protein for use in methods according to the invention may be modified, in sequence or otherwise, in order to change their activity or alter their degradation characteristics.

The invention further includes methods wherein at least one of the transcription factor specific for pancreatic cells and the activating means is provided as a protein which has been introduced into the cell by any suitable delivery means such as by the use of liposomes or electroporation.

The transformed cell can also be examined for the development of pancreatic cell phenotype. For example, expression of insulin could be detected by PCR, northern blot, immunocytochemistry, Western blot, RIA or ELISA. Alternatively a marker gene such as green florescent protein could be used for identification or selection of differentiated pancreatic cells. Methods for engineering a host cell for expression of a desired gene product(s) and implantation or transplantation of the engineered cells (e.g., ex vivo therapy) are known in the art.

In general, after expansion of the transformed or treated cells in vitro, the cells are implanted into the mammalian subject by methods well known in the art. The number of cells to be transplanted can be determined based upon such factors as the levels of polypeptide expression achieved in vitro, and/or the number of cells that survive implantation. The transformed cells are preferably implanted in an area of dense vascularization such as the liver, and in a manner that minimizes surgical intervention in the subject. The engraftment of the implant of transformed cells is monitored by examining the mammalian subject for classic signs of graft rejection, i.e., inflammation and/or exfoliation at the site of implantation, and fever, and by monitoring blood glucose levels.

The precise amount of pancreatic transcription factor-encoding nucleic acid administered will vary greatly according to a number of factors including the susceptibility of the target cells to transformation, the size and weight of the subject, the levels of protein expression desired, and the condition to be treated. The amount of nucleic acid and/or the number of infectious viral particles effective to infect the targeted tissue, transform a sufficient number of cells, and provide for production of a desired amount of pancreatic tissue can be readily determined based upon such factors as the efficiency of the transformation in vitro and the susceptibility of the targeted cells to transformation. For example, the amount of DNA introduced into the liver of a human is, for example, generally from about 1 mug to about 750 mg, preferably from about 500 micrograms to about 500 mg, more preferably from about 10 mg to about 200 mg, most preferably about 100 mg. Generally, the amounts of DNA can be extrapolated from the amounts of DNA effective for delivery and expression of the desired gene in an animal model. For example, the amount of DNA for delivery in a human is roughly 100 times the amount of DNA effective in a rat.

Regardless of whether the pancreatic transcription factor-encoding nucleic acid is introduced in vivo or ex vivo, the nucleic acid (or protein-containing composition or pancreatic cells produced in vitro or recombinant cells expressing the pancreatic transcription factor nucleic acid that are to be transplanted for development into pancreatic tissue in vivo post-transplantation) can be administered in combination with other genes and other agents.

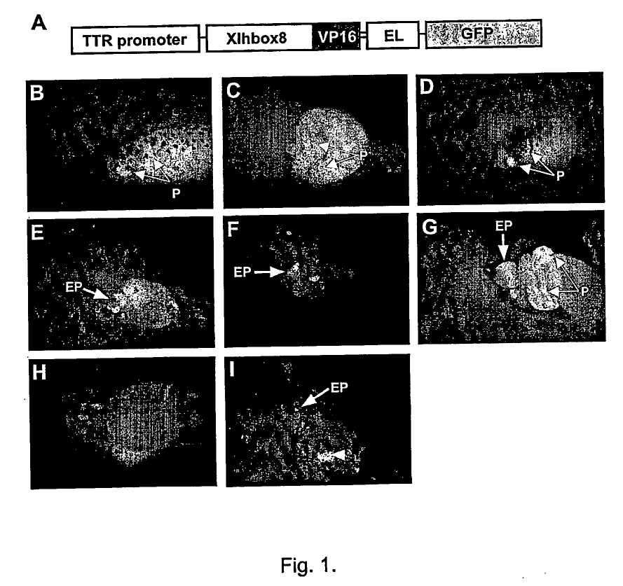

The present invention will now be described, by way of example only, with reference to the following non-limiting examples, illustrated by FIGS. 1 to 11 in which endogenous pancreas is labelled P and ectopic pancreas is labelled EP:

FIG. 1A shows a schematic representation of the transgene construct X1Hbox8-VP16 which may be used according to the present invention.

FIGS. 1B to 1I show the results of transfection of Xenopus with a construct comprising the 3 kb liver specific promoter TTR used to over-express X1Hbox8-VP16 in the liver after the liver has already formed. The 200 bp pancreas specific elastase promoter driving GFP expression is used to distinguish the transgenic tadpoles and easily identify the ectopic pancreatic tissue in the liver. (B,C) showing elastase GFP transgenic tadpoles at stage 40 and stage 42, (D-I) showing TTR-X1Hbox8; EL-GFP transgenic tadpoles. (D) stage 40. GFP fluorescence only seen in the pancreas. (E-I) stage 45-48 tadpoles. (E) Half of the liver is expressing GFP. The dorsal and ventral buds are visible on either side of the gut tube. (F) Several prominent spots of GFP are apparent within the liver. The GFP expression in the normal pancreas is seen at the bottom of the picture. (G) Ectopic GFP is seen in the liver near the heart. The dorsal and ventral pancreas is located posterior to the liver. (H) Ectopic GFP fluorescence is seen in anterior position adjacent to the heart. (I) Ectopic GFP can be seen underneath the gall bladder. The normal pancreas can be seen in the middle of the gut.

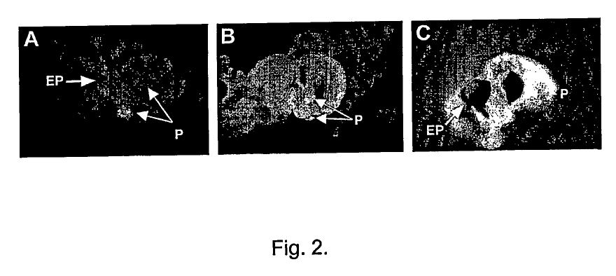

FIG. 2 shows ectopic amylase expression in the liver of transgenic tadpoles. All three pictures are from the same tadpole. (A) Live image of TTR-X1Hbox8-VP16:EL-GFP transgenic tadpole, showing ectopic expression (labelled EP) of elastase-GFP in the transdifferentiated liver, anterior to the normal pancreas. (B) After dissection, the transdifferentiated liver can be seen opposite the normal pancreas. Notice the lack of the GFP in the centre of the liver (highlighted with dashed white line). (C) Amylase expression. Amylase RNA is detected in same place as GFP. Amylase expression is seen in the transdifferentiated liver (labelled EP) as well as in the normal pancreas (labelled P).

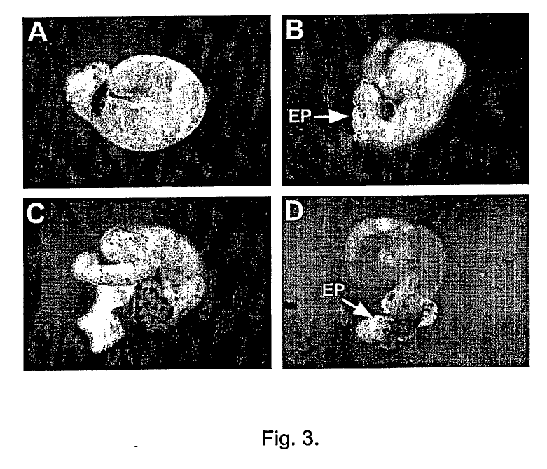

FIG. 3 shows that endocrine genes are expressed in the transdifferentiated liver. (A,B) Insulin RNA expression (C,D) Glucagon RNA expression. (A) Wild type insulin expression.

Expression is only seen in the dorsal pancreas at this stage in several prominent clusters. (B) TTR-X1Hbox8-VP16 transgenic gut. Several ectopic clusters of insulin expression are seen in the liver opposite the pancreas. (C) Wild type glucagon expression. Expression is seen in the several spots within the pancreas as well as in the stomach and the intestine. (D) TTR-X1Hbox8-VP16 transgenic gut. Ectopic glucagon expression is found in the transdifferentiated liver (labelled EP) opposite the normal pancreas.

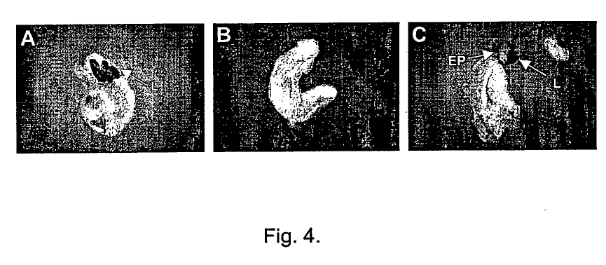

FIG. 4 shows that liver gene expression is decreased in transgenic guts. (A) Wild type expression of transthyretin (TTR) RNA. Expression is only seen within the liver (labelled L). (B,C) TTR-X1Hbox8-VP 16 transgenic guts. (B) In this example no TTR expression can be seen in the whole gut. (C) TTR expression is seen only in one half of the liver (labelled L). The other half has transdifferentiated into pancreas (labelled EP).

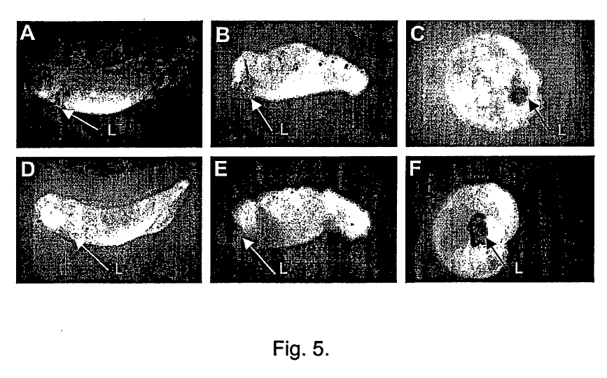

FIG. 5 shows the timing of liver differentiation. (A-C) Xhex RNA expression. (A) At stage 35 Xhex RNA is detected in a region just behind the heart. (B) By stage 40, the liver is a separate organ and Xhex RNA is only found within the liver (labelled L). (C) At stage 45 the expression of Xhex is the same as at stage 40 being expressed throughout the entire liver (labelled L). (D-F) AMBP RNA expression. (D) AMBP is first detected in the liver (labelled L) at stage 35 as seen with Xhex. (E,F) At stages 40 and 45 respectively AMBP expression is found throughout the entire liver (labelled L).

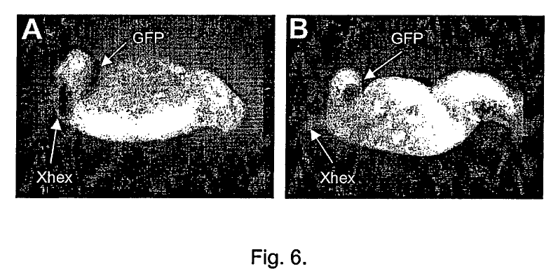

FIG. 6 shows that liver differentiation begins normally in TTR-X1Hbox8-VP16 tadpoles. (A,B) Xhex (blue) and GFP (magenta-driven by the elastase promoter) expression. GFP RNA is seen specifically in the dorsal and ventral pancreas at stage 40; no GFP expression is detected in the liver. Xhex expression is normal at this stage being present throughout the whole liver.

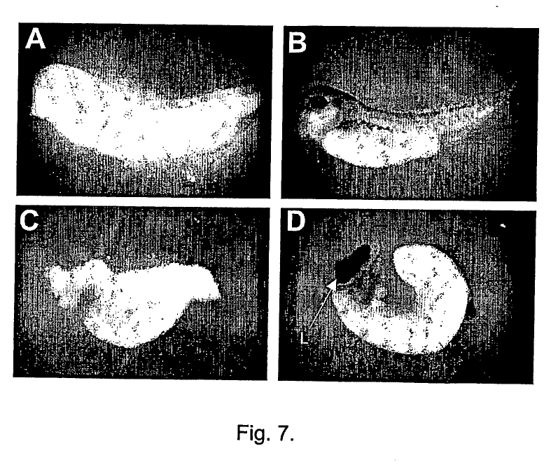

FIG. 7 shows the timing of TTR promoter activity in Xenopus tadpoles. RNA in situ hybridization for GFP RNA driven by the TTR promoter. No expression of GFP is detected at stages (A) 32, (B) 39, (C) 41. (D) Expression is first detected in the liver (labelled L) beginning at stage 44.

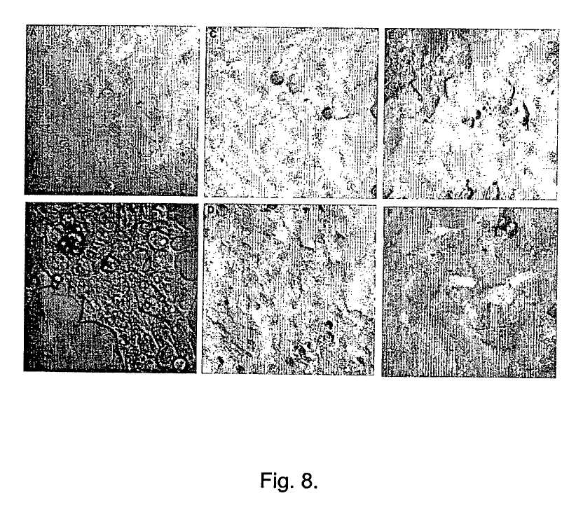

FIG. 8 shows the GFP staining of cells transfected with constructs: (A) B13 transfected with CS2-X1Hbox8-VP16; Elas-GTP, (B) FAO transfected with pcDNA3-Elas-GFP, (C) FAO transfected with nuc-GFP, (D) FOA transfected with TTR-GFP and (E,F) FAO transfected with CS2-XVEG.

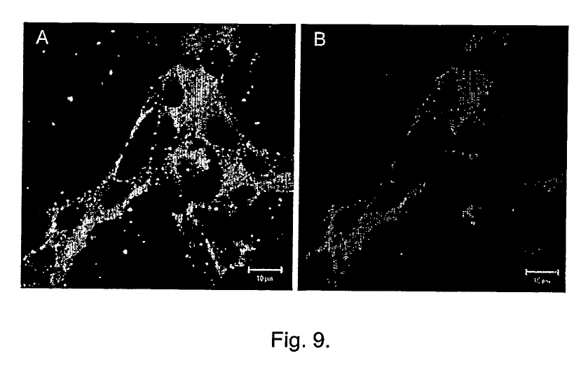

FIG. 9 shows HepG2 (human hepatoma) cells transfected with TTR-VP16-X1HBOX8; ElasGFP and incubated for 5 days and then fixed. (A) GFP expression, (B) immunostained for insulin. In this field some, but not all, of the GFP positive cells are also positive for insulin.

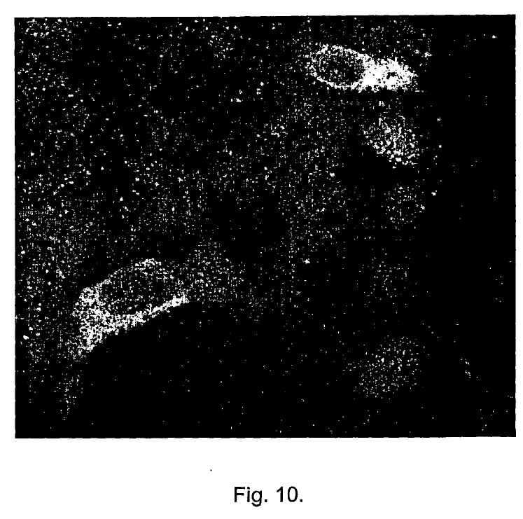

FIG. 10 shows HepG2 (human hepatoma) cells transfected with TTR-VP 16-Neurogenin3; ElasGFP and incubated for 5 days before being fixed and immunostained for insulin.

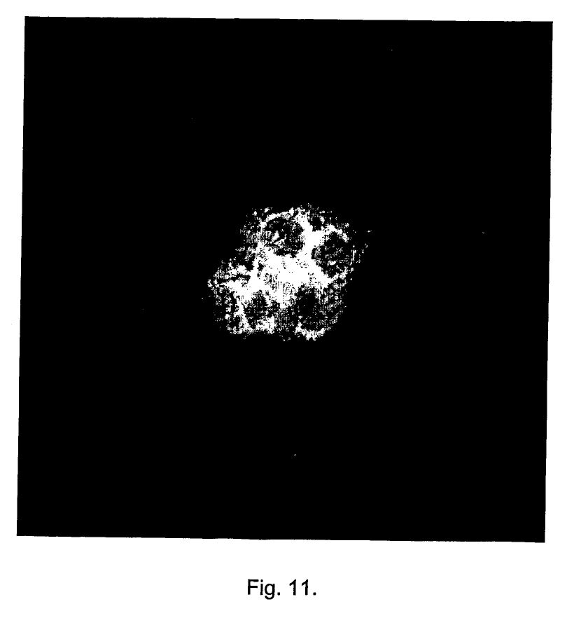

FIG. 11 shows HepG2 (human hepatoma) cells transfected with TTR-p48-VP16; ElasGTP and incubated for 5 days before being fixed and immunostained for amylase.

### EXAMPLES

### Example 1

### Xenopus In Vivo

Transgenesis

Transgenic Xenopus laevis tadpoles were made according to Kroll & Amaya (1996) Development 122:3173-83 with slight modifications according to Beck & Slack (1999) Mech Dev. 88:221-7, except for the following two changes. First, the high speed cytoplasmic egg extract was heat treated for 8 minutes at 80° and recentrifuged at 70,000 rpms for 10 minutes. 10 μl of the cytoplasmic extract was then used for each reaction. Second, no restriction enzymes were included in the incubation of sperm and transgene DNA. 5-10 μg of transgene DNA was linearized with either SacII or NotI, extracted with phenol/chloroform and ethanol precipitated. The DNA was resuspended to a final concentration of 0.5 μg/μl; 0.5 μg of DNA was used to make transgenics.

To visualize GFP fluorescence, tadpoles were anaesthetized in 1/200 MS-222 (3-aminobenzoic acid ethyl ester, Sigma) and visualized using a Leica Fluo III fluroescent-dissecting microscope.

Transgene Constructs

TTR-X1Hbox8-VP16:EL-GFP was made as follows:

Full length X1Hbox8 was PCR isolated from X1Hbox8-CS2 (as described in Wright et al., (1988) Development 104, 87-794) using the SP6 promoter and a 3′ primer with a ClaI site having the sequence shown as SEQ ID NO. 1.

SEQ ID NO. 1

5′-TTT ATC GAT TTC TGC CTG CC-3′.

It was cut with ClaI and subcloned into the ClaI site of VP16-N (as described in Kessler, D S (1997) PNAS 94, 13017-13022). The 3 kb TTR promoter (as described in Yan et al., EMBO J. 9, 869-878) was cut with BamHI and filled in with Klenow and cloned upstream of X1Hbox8-VP16 in the Hind III (blunt) site. Lastly, the Elastase-GFP (as described in Kruse et al., (1993) Genes Dev. 7, 774-786) was cut with NotI and cloned into the NotI site of TTR-X1Hbox8-VP16.

The resultant plasmid, TTR-X1Hbox8-VP16:EL-GFP is 10.5 kb. For TTR-GFP and El-GFP, transgene DNA was cut as in Beck & Slack (1999) supra.

Whole Mount In Situ Hybridization

Whole mount in situ hybridization was performed according to Harland (1991) Methods Cell Biol. 36:685-95 using digoxygenin and fluorscein labelled probes. Xenopus amylase was cloned by degenerate PCR as in Horb & Slack (2002) Mech Dev, 113, 153-157 into pCR Script. For antisense RNA it was linearized with EcoRV and transcribed with T3. Xenopus insulin was cloned by PCR based on published sequence (nucleotides 3-752) into pCR Script and was linearized with EcoRI and transcribed with T3. Xenopus glucagon was cloned by PCR based on published sequence (nucleotides 1-1151) into pCR Script and was linearized with NotI and transcribed with T7. Xenopus transthyretin (TTR) was cloned by PCR based on published sequence (nucleotides 10-500) into pCR Script and was linearized with EcoRI and transcribed with T3. Xenopus hex (Xhex) (kind gift of P. Krieg) was linearized with NotI and transcribed with T7. Xenopus alpha1-microglobulin/bikunin (AMBP) (kind gift of A. Kawahara) was linearized with SacI and transcribed with T7. For double-in situ hybridization, fluroescein labelled probes were detected first with 175 μg/ml magenta phosphate (5-bromo-6-chloro-3-indolyl phosphate, Molecular Probes Europe), heat inactivated and then the digoxygenin labelled probes were detected with 175 μg/ml BCIP.

Results

Previous attempts to induce ectopic pancreatic tissue in various regions of the gut by overexpressing PDX-1 have met with little success. In order to transdifferentiate the liver into pancreas, we have used the newly developed transgenic technique in Xenopus to overexpress the Xenopus homologue of PDX-1, Xhlhbox8, in the developing liver. Previously, our lab has shown that certain mammalian gut promoters would drive tissue specific expression of GFP. One of these promoters, transthyretin (TTR) from mouse, displayed liver-specific expression at a stage when the liver has already differentiated. The TTR promoter was here used to ectopically express X1Hbox8 in the liver. To make it easier to identify the transgenic tadpoles and to monitor the efficiency of transdifferentiation, the elastase promoter was used to drive GFP expression (EL-GFP) and was included in the same transgene (FIG. 1A). The elastase promoter used is a 200 bp element that drives expression in both endocrine and exocrine cells. As shown previously in our lab, the elastase promoter drives expression in both the dorsal and ventral pancreatic buds at stage 41 (FIG. 1B). At stage 42, after the dorsal and ventral pancreatic buds have fused, EL-GFP expression can be seen posterior to the heart and liver on either side of the duodenum (FIG. 1C).

In our first experiment we overexpressed the full-length unmodified form of X1Hbox8 in the liver using the TTR promoter. This experiment however, was unsuccessful: no ectopic expression of endocrine or exocrine markers was detected in the liver (data not shown). Since it is known that transcription factors require protein-protein interactions and an open chromatin state to function properly, we hypothesized that the overexpression of X1Hbox8 in the liver did not result in ectopic pancreatic tissue because the necessary cofactors were not present within the liver. To circumvent this problem we produced a modified form of X1Hbox8 by fusing the VP16 activation domain to the full length X1Hbox8 (FIG. 1A). VP16 is known to function as a strong transactivator and to open up chromatin and has been used previously in Xenopus to determine whether transcription factors function as activators or repressors.

Overexpression of the modified form of X1Hbox8, X1Hbox8-VP16, in the liver results in the trandifferentiation of liver to pancreas. Initially, liver and pancreas development occurred normally in transgenic tadpoles containing TTR-X1Hbox8-VP16. Elastase-GFP is only expressed in the usual place, i.e. the dorsal and ventral pancreatic buds (FIG. 1D). No ectopic EL-GFP expression is seen until stage 45 (see below). In Xenopus transgenics, each transgenic animal represents a separate integration event, with differing amounts of DNA integrating into different places in the host genome. As a result, in TTR-X1Hbox8-VP16 transgenics, we find a variety of phenotypes, from partial to complete transdifferentiation (FIG. 1E-I). In all cases it is easy to distinguish the endogenous pancreas expressing GFP from the ectopic pancreatic tissue. When the whole liver transdifferentiates into pancreas, EL-GFP expression is seen throughout the entire liver, anterior to the endogenous pancreatic expression (FIG. 1E,F). In other cases only half the liver is transdifferentiated (FIG. 1G). When only a portion of the liver is converted to pancreas, large clusters of EL-GFP fluorescence is detected in various parts of the liver (FIG. 1H,I). In all cases the rest of the tadpole appears normal.

It can be argued that the use of a small 200 bp element from the elastase promoter to measure the conversion of liver to pancreas is artificial and that the GFP expression only represents activation of the promoter and not a true conversion to a pancreatic fate. To unequivocally determine whether the ectopic EL-GFP expression represents true conversion of liver to pancreas, we also examined the RNA expression of several exocrine and endocrine markers. To determine whether exocrine cells are present in the ectopic pancreatic tissue, we examined the expression of amylase RNA in the TTR-X1Hbox8-VP16 transgenic guts (FIG. 2). We have recently cloned the Xenopus amylase cDNA and have shown that its expression coincides both spatially and temporally with two other exocrine markers, trypsinogen and elastase. As shown in FIG. 2, amylase RNA is expressed in the ectopic pancreatic tissue in identical regions to the EL-GFP expression. Ectopic EL-GFP fluorescence in the liver can be seen clearly in the live tadpole (FIG. 2A) as well as in the dissected tadpole (FIG. 2B). In this example most of the liver has been transdifferentiated into pancreas, but a small portion in the centre does not express GFP (FIG. 2A,B). In agreement with the EL-GFP, ectopic amylase RNA is detected throughout almost the whole liver on the opposite side to the normal amylase expression (FIG. 2C). No expression of amylase RNA is detected in the middle of the liver. This is identical to that seen with EL-GFP. The ectopic expression of amylase RNA shows that the EL-GFP expression represents true pancreatic tissue and not just promoter activation.

We have also examined insulin and glucagon expression to determine whether the ectopic pancreatic tissue included endocrine cells. In wild type guts insulin RNA is only detected in the dorsal pancreas in several large clusters (FIG. 3A). In TTR-X1Hbox8-VP16 transgenic guts, ectopic insulin is detected in the liver as well as the normal endogenous expression (FIG. 3B). Glucagon expression is normally found in small groups of cells in the pancreas beginning at stage 45 (FIG. 3C). Expression is also detected in the stomach and small intestine. In the TTR-X1Hbox8-VP16 transgenic tadpoles ectopic glucagon expression can be seen within the liver (FIG. 3D arrow). These results demonstrate that cells expressing endocrine genes are also present in the transdifferentiated liver.

Liver Gene Expression

To determine whether the ectopic pancreatic tissue still expresses liver markers we examined the transgenic guts for expression of endogenous Xenopus transthyretin RNA. At stage 45 in wild type guts, TTR RNA is only detected within the liver (FIG. 4A). In agreement with the various phenotypes described previously, we find several different levels of TTR expression in TTR-X1Hbox8-VP16 transgenic guts. When the whole liver is converted to pancreas, no expression of TTR is detected (FIG. 4B). However, when only half the liver is transdifferentiated, we find TTR expression in the half that has not been converted to pancreas (FIG. 4C). This is in agreement with FIG. 1G that shows EL-GFP expression in only half of the liver. These results demonstrate that upon transdifferentiation of the liver to pancreas, endogenous liver markers are quickly turned off.

It can be argued that our use of the term transdifferentiation is incorrect because we are using embryonic tadpoles. However as shown in FIGS. 5-7 we believe that the term transdifferentiation is correct. Differentiation is defined as “the synthesis by a cell of species of protein different to those made at an earlier developmental stage, or different to those made by surrounding cells at the same stage”. For example, liver differentiation markers include AMBP, Xhex, TTR and albumin, while pancreas differentiation markers include amylase, insulin and glucagon. Transdifferentiation is therefore the conversion of one differentiated cell into another differentiated cell. To determine when liver differentiation occurs in Xenopus we examined the expression of two liver markers, Xhex and AMBP, in the developing tadpole. Previous work by others has shown that liver differentiation markers begin to be expressed at stage 32. Xhex is a homeobox gene that is expressed in the developing liver and has been shown to play an important role in liver development. AMBP is short for alpha1-microglobulin/bikunin precursor and it is expressed in liver parenchymal cells. At stage 35, both of these markers are specifically expressed in a region just posterior to the heart marking the future liver (FIG. 5A,D). By stage 40/41 the liver has already formed a separate organ and expression of Xhex and AMBP is seen within the liver (FIG. 5B,E). This liver-specific expression persists into later stage (FIG. 5C,F). These results demonstrate that by stage 40/41 the liver has differentiated and formed a separate organ.

To confirm that liver differentiation does indeed occur normally in TTR-X1Hbox8-VP16 transgenic tadpoles, we examined the spatial expression of Xhex in these animals. To confirm that the dissected guts were indeed transgenic we also stained for the expression of gfp RNA, which is controlled by the elastase promoter. In TTR-X1Hbox8-VP16 transgenic guts at stage 40/41, Xhex is detected in the liver (FIG. 6). These same transgenic guts were stained for gfp RNA expression to mark the border of the pancreas. No ectopic gfp is detected in the liver, but expression is clearly seen within the pancreas (FIG. 6). In contrast, in later stage transgenic guts, ectopic gfp expression can be detected in the liver once the liver has been converted to pancreas (see FIG. 1). These results thus demonstrate that the liver does differentiate normally in TTR-X1Hbox8-VP16 transgenic tadpoles.

To further confirm that overexpression of X1Hbox8-VP16 in the liver only occurs after the liver has differentiated we examined the time course of activation of the TTR promoter, used to drive expression of X1Hbox8-VP16, in TTR-GFP transgenic tadpoles. No expression of gfp RNA is detected during the early stages of liver differentiation (FIG. 7A-C). Low levels of gfp RNA are first detected within the liver beginning at stage 43 (FIG. 7D). As shown above, by this stage the liver has already differentiated. These results demonstrate that the TTR promoter is not active until stage 43 and that ectopic expression of X1Hbox8-VP16 in the liver does not occur until after the liver differentiates.

### Example 2

### Liver Cells In Vitro

FAO hepatoma cells are a well-differentiated liver cell line. Using these cells as a model system we transfected various constructs encoding the Xenopus homologue (X1Hbox8) of the mammalian pancreatic transcription factor, PDX-1. The results suggest that the hepatocytes can be turned into pancreatic cells.

Transfection of Mammalian Cells

FAO rat hepatoma cells deposited under accession number ECACC 8904271 were obtained from the ECACC (CAMR, Salisbury, U.K.) and cultured in Coon's medium (Sigma Chemical Co, Poole, U.K.) in 35 mm dishes. The rat pancreatic cell line AR42J deposited under accession number ECACC 93100618 was cultured as described in Shen et al., (2000) Nature Cell Biology 2, 879-887.

The TTR-X1Hbox8-VP16:EL-GFP plasmid was transfected into the FAO cells or AR42J-B13 cells using the ExGen 500 in vitro transfection reagent (Fermentas, Italy) according to the manufacturer's instructions. Essentially, 6.61 μl of transfection reagent was added for 2 μg of plasmid DNA. Other plasmids were also transfected to control for the leakiness of the elastase promoter and to test the expression of TTR alone. These included, the −3 kb TTR promoter driving GFP, the elastase promoter driving GFP and the constitutive CMV promoter driving GFP. GFP has a nuclear localisation sequence inserted upstream so all images show nuclei either without or with GFP fluorescence.

Results

The construct designed in Example 1 (TTR-X1Hbox8-VP16:-EL-GFP) was transfected into FAO cells as described above. 3-4 days later cells begin to express GFP indicating that markers of pancreatic exocrine expression are now being induced in hepatocytes (FIGS. 8E & F). We also performed various control transfection experiments e.g. to test the leakiness/stringency of the elastase-GFP. Firstly, the FAO cells expressed nucGFP from the constitutive CMV promoter in the plasmid pcDNA3-nuc-GFP (FIG. 8C), we could also drive nuc-GFP expression from the hepatic transthyretin (TTR) promoter indicating that this liver gene is expressed and the correct machinery exist for transactivating the gene (FIG. 8D). This also proves that the TTR-promoter being used for driving X1Hbox8 functions in FAO cells.

To demonstrate that the elastase-GFP was not expressed in FAO cells we transfected a plasmid pcDNA3-elastase-promoter-GFP into FAO cells. There were no GFP-positive cells (FIG. 8B), despite the laser intensity being very high (hence the green hue in FIG. 8B). This result clearly shows that the initial result is not random expression of the elastase-GFP. Lastly, we assume that the Elas-promoter-GFP portion of the construct CS2-TTR-promoter-X1Hbox8-VP16-Elas-promoter-GFP should be active in pancreatic cells. To determine if this was the case, we transfected in the CS2-TTR-promoter-X1Hbox8-VP16-Elas-promoter-GFP construct to AR42J-B13 cells which we know already expresses the exocrine phenotype. GFP-positive cells were obtained within 24 hr and the image was collected at 48 hr after transfection (FIG. 8A).

### Example 3

### Glucose-Stimulated Insulin Release from Ectopic Pancreas in Xenopus

Tissue explants were dissected from control Xenopus tadpoles transgenic for TTR-X1Hbox8-VP16; Elas-GFP. They were cultured for 3 hours in 50 μl of 70% L15 medium with or without 30 mM glucose. Insulin was measured using a Mercodia test kit (order number 10-1113-01, Mercodia AB, Uppsala, Sweden). Results shown below (table 1) demonstrate that whilst both endogenous and ectopic pancreas releases insulin in response to glucose stimulation, untransdifferentiated liver does not.

Results

TABLE 1

| Tissue Sample | Insulin release (units/explant) |
|---|---|
| 8 d pancreas | 0.009 |
| 8 d pancreas + glucose | 0.363 |
| 10 d pancreas | 0.011 |
| 10 d pancreas + glucose | 0.444 |
| 10 d ectopic pancreas + glucose | 0.216 |
| 10 d liver | 0.046 |
| 10 d liver + glucose | 0.017 |

Calibration:

| Insulin concentration (μg/l) | Units |
|---|---|
| 0.02 | 0.047 |
| 0.05 | 0.062 |
| 0.15 | 0.113 |
| 0.40 | 0.334 |
| 1.0 | 1.132 |

### Example 4

### Insulin Producing Cells are Produced by Transdifferentiation

HepG2 (human hepatoma) cells were transfected with TTR-X1HBOX8-VP16; El-GFP and incubated for 5 days. They were fixed and examined for GFP (green) (FIG. 9A) and immunostained for insulin (red) (FIG. 9B).

This example provides evidence that many, if not all, GFP positive cells (i.e. those that have been transfected) are also positive for insulin.

### Example 5

### Alternative Pancreas Specific Transcription Factors May be Used for Transdifferentiation

To demonstrate that human liver cells may be transdifferentiated to ectopic pancreas with transcription factors other than PdX1/X1Hbox8, HepG2 (human hepatoma) cells were transfected with TTR-VP16-Neurogenin 3; Elastase-GFP or with TTR-VP16-p48; Elastase-GFP.

Neurogenin 3 is known to be necessary for development of endocrine cells in the pancreas and p48 development (see M E. Wilson, D. Scheel, M S. German. Gene expression cascades in pancreatic development. Mechanisms of Development 120:65-80(2003)). The methods used were the same as used for HepG2 experiments already described and the names of the constructs used above reflect the fact that the VP16 was located at the N terminal of the transcription factor gene.

Cells were cultured for 5 days following transfection and then fixed and immunostained for insulin (FIG. 10) or amylase (FIG. 11). The results indicated that the methods of the invention do not require the use of the Pdx1/x1hbox8 to be successful and that the VP16 transcriptional activator can be placed in the construct towards the N or C terminus of at least the transcription factors investigated with equal success.

1. A method for converting non-pancreatic cells into pancreatic cells, the method comprising providing to non-pancreatic cells a transcription factor specific for pancreatic cells, in the presence of an activating means able to activate the transcription factor, such that the cells in which the transcription factor is expressed convert into pancreatic cells.

2. A method according to claim 1 in which the transcription factor is provided as a protein.

3. A method according to claim 1 in which the transcription factor is provided by way of expression of a nucleic acid in the cells to which it is provided.

4. A method according to any of claims 1 to 3 in which the non-pancreatic cells are differentiated cells that constitute part of a tissue or organ.

5. A method according to claim 4 in which the non-pancreatic cells constitute part of an endodermal organ.

6. A method according to claim 4 in which the non-pancreatic cells comprise cells of the liver, lung, thyroid or intestine.

7. A method according to claim 1 in which the activating means comprises a exogenous protein.

8. A method according to claim 1 in which the activating means comprises a heterologous nucleic acid molecule.

9. A method according to claim 7 or claim 8 in which the activating means comprises the transcriptional activation domain VP16 from Herpes simplex virus and functional homologues thereof.

10. A method according to claim 1 in which the activating means causes ectopic expression and activity of an endogenous transcription factor gene.

11. A method according to claim 1 in which the activating means causes ectopic expression and activity of an exogenous transcription factor gene.

12. A method according to claim 11 in which the exogenous transcription factor gene is introduced into the cell as a gene fusion with nucleic acid encoding an activating domain.

13. A method according to claim 12 in which the gene fusion comprises VP16 fused to a pancreatic transcription factor.

14. A method according to claim 13 in which the activating domain comprises a sub-fragment of VP16 comprising the C-terminal 78 amino acids.

15. A method according to claim 1 in which the provision of the pancreatic transcription factor is targeted to cells of a particular tissue.

16. A method according to claim 15 in which the pancreatic transcription factor is provided to the cells of a particular tissue under the control of a tissue specific promoter to allow targeted expression of the transcription factor in that particular tissue.

17. A method according to claim 16 in which the pancreatic transcription factor is expressed in liver cells under the control of a liver specific promoter

18. A method according to claim 17 in which the liver specific promoter is the transthyretin (TTR), glucose 6 phosphatase or albumin promoter.

19. A method according to claim 16 in which the pancreatic transcription factor is expressed in intestinal cells under the control of a promoter specific to intestinal cells.

20. A method according to claim 19 in which the intestine specific promoter is the intestinal fatty acid binding protein (IFABP) promoter.

21. A method according to claim 16 in which the pancreatic transcription factor specific for pancreatic cells may be expressed in lung cells under the control of a promoter specific to lung cells.

22. A method according to claim 21 in which the lung specific promoter is the surfactant A or surfactant C promoter.

23. A method according to claim 16 in which the pancreatic transcription factor specific for pancreatic cells may be expressed in thyroid cells under the control of a promoter specific to thyroid cells.

24. A method according to claim 23 in which the thyroid specific promoter is the thyroglobulin promoter.

25. A method according to claim 1 in which the means for providing to non-pancreatic cells a transcription factor for pancreatic cells comprises transfection of the non-pancreatic cells with a nucleic acid construct comprising an activating domain and an isolated pancreatic transcription factor gene, whose expression is under the control of a tissue specific promoter.

26. A method according to claim 1 in which the pancreatic transcription factor provided to non-pancreatic cells is the pancreatic transcription factor PDX1 (mouse) or homologues thereof, selected from X1HboxS (xenopus), STF-1 (rat), and IPF-1 (human).

27. A method according to claim 1 in which the pancreatic factor provided to non-pancreatic cells is the pancreatic transcription factor neurogenin 3 (human) or a homologue thereof.

28. A method according to claim 1 in which the pancreatic transcription factor provided to non-pancreatic cells is the pancreatic transcription factor p48 (human) or a homologue thereof.

29. A method according to claim 3 in which said nucleic acid is provided to said cells via in vivo or ex vivo gene therapy.

30. A method according to claim 1 which is carried out in vitro.

31. A method according to claim 1, wherein the provision of either the transcription factor specific for pancreatic cells or the provision of the activating means, or the provision of both the transcription factor specific for pancreatic cells and the activating means, is transient.

32. A nucleic acid construct comprising a pancreatic transcription factor and an activating domain that is capable of activating the pancreatic transcription factor in cells into which the nucleic acid construct is introduced.

33. A nucleic acid construct according to claim 32 in which the pancreatic transcription factor comprises PDX-1 (mouse) or homologues thereof, selected from X1Hbox8 (xenopus), STF-1 (rat), and IPF-1 (human).

34. A nucleic acid construct according to claim 32 in which the pancreatic transcription factor comprises neurogenin 3 (human) or a homologue thereof.

35. A nucleic acid construct according to claim 32 in which the pancreatic transcription factor comprises p48 (human) or a homologue thereof.

36. A nucleic acid construct according to claim 32 or claim 35 in which the activating domain comprises the transcriptional activation domain VP16 from Herpes simplex virus or functional homologues, analogues and derivatives thereof which maintain VP16 activity.

37. A nucleic acid construct claim 32 further comprising a promoter.

38. A nucleic acid according to claim 37, wherein the promoter is not constitutively active.

39. A nucleic acid construct according to claim 37 or claim 38 in which the promoter is tissue specific.

40. A nucleic acid construct according to claim 39 in which the promoter is the transthyretin (TTR), glucose 6 phosphatase, albumin, intestinal fatty acid binding protein, thyroglobulin, surfactant A or surfactant C promoter.

41. A nucleic acid construct according to claim 32 further comprising a reporter gene.

42. A nucleic acid construct according to claim 41 in which the reporter gene is green fluorescent protein (GFP).

43. A nucleic acid construct according to claim 42 in which the GFP reporter gene is under the control of an elastase promoter, which is capable of driving expression in both endocrine and exocrine cells.

44. A nucleic acid construct according to claim 42 in which the GFP reporter gene is under the control of a promoter which is capable of driving expression in specific pancreatic cells.

45. A nucleic acid construct comprising a TTR promoter operably linked to a X1Hbox8 gene and VP16, linked to an elastase promoter controlling expression of GFP.

46. A vector comprising the nucleic acid construct according to claim 32 or claim 41.

47. A composition comprising a protein transcription factor specific for pancreatic cells or a nucleic acid encoding a transcription factor specific for pancreatic cells, said composition further comprising an activating means capable of activating the transcription factor in non-pancreatic cells wherein the activating means is a protein and/or the composition comprises a protein transcription factor specific for pancreatic cells.

48. A composition according to claim 47 in which said transcription factor specific for pancreatic cells is selected from PDX1 (mouse), X1HboxS (xenopus), STF-1 (rat), and IPF-1 (human) and the activating means causes ectopic expression and activity of an endogenous transcription factor gene.

49. Non-pancreatic cells transformed with the nucleic acid construct according to claim 32 or treated with the composition according to claim 47.

50. Pancreatic tissue of non-pancreatic origin produced by the method according to claim 1, or by transformation of non-pancreatic cells with the nucleic acid construct of claim 32, or by the treatment of non-pancreatic cells with the composition according to claim 47, or by culturing cells according to claim 49.

51. A method of treatment of pancreatic disorders comprising converting non-pancreatic cells into pancreatic tissue.

52. A method according to claim 51 wherein said disorder is diabetes or pancreatic cancer or another pancreatic disease.

53. A method according to claim 52 wherein said disorder is type 1 or insulin dependent type 2 diabetes.

54. A method according to claim 51, wherein said converting is accomplished by treating non-pancreatic cells with a nucleic acid construct comprising a pancreatic transcription factor and an activating domain that is capable of activating the pancreatic transcription factor in cells into which the nucleic acid construct is introduced or by treating non-pancreatic cells with a composition comprising a protein transcription factor specific for pancreatic cells or a nucleic acid encoding a transcription factor specific for pancreatic cells, said composition further comprising an activating means capable of activating the transcription factor in non-pancreatic cells wherein the activating means is a protein and/or the composition comprises a protein transcription factor specific for pancreatic cells.

55. A pharmaceutical composition comprising (a) a composition of matter selected from the group consisting of a nucleic acid construct according to claim 32 and a composition according to claim 47, and (b) a carrier.

56. A pharmaceutical composition comprising (a) cells according to claim 49 and (b) a carrier.

57. A method of treating diabetes or pancreatic cancer or another pancreatic disease comprising administering to a patient in need the pharmaceutical composition according to claim 56.

58. The method according to claim 57, wherein said diabetes is type 1 diabetes or insulin dependent type 2 diabetes.

59. A method of treatment of pancreatic disorders comprising implanting into a subject suffering from a pancreatic disorder the cells according to claim 49.

60. A method according to claim 59 wherein said pancreatic disorder is diabetes or pancreatic cancer.

61. A method according to claim 60 wherein said diabetes is type 1 or insulin dependent type 2 diabetes.
